(19) United States
(12) Patent Application Publication
(10) Pub. No.: US 2013/0053569 A1
(43) Pub. Date: Feb. 28, 2013

(54) PROCESS FOR THE PREPARATION OF PRASUGREL HCL SALT

(71) Applicants: Jayaraman Venkat Raman, Vadodara (IN); Samir Patel, Vadodara (IN); Abhijit Kadam, Vadodara (IN); Vijay Patel, Vadodara (IN); Jignesh Patel, Vadodara (IN)

(72) Inventors: Jayaraman Venkat Raman, Vadodara (IN); Samir Patel, Vadodara (IN); Abhijit Kadam, Vadodara (IN); Vijay Patel, Vadodara (IN); Jignesh Patel, Vadodara (IN)

(21) Appl. No.: 13/579,763

(22) PCT Filed: Mar. 17, 2011
(86) PCT No.: PCT/IB11/51110

§ 371 Date: Nov. 13, 2012

(30) Foreign Application Priority Data
Mar. 23, 2010 (IN) 784/MUM/2010

(51) Int. Cl. C07D 495/04

(52) U.S. Cl. 546/114

(57) ABSTRACT

The present invention relates to process for the preparation of Prasugrel HCl having formula (I).

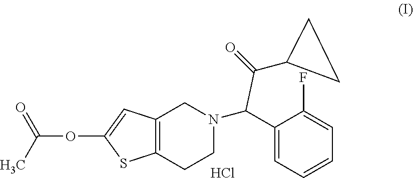 [SMILES: CC(=O)Oc1cc2c(s1)CCN(C(C(=O)C1CC1)c1ccccc1F)C2 ; Cl]

### FIELD OF THE INVENTION

The present invention relates to process for the preparation of Prasugrel HCl having formula (I).

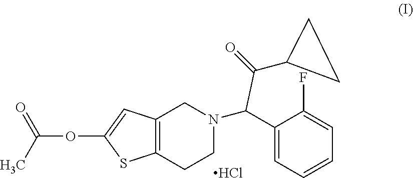 [SMILES: CC(=O)Oc1cc2c(s1)CCN(C(C(=O)C1CC1)c1ccccc1F)C2 ; Cl]

### BACKGROUND OF THE INVENTION

Prasugrel HCl is chemically known as 5-[2-cyclopropyl-1-(2-fluorophenyl)-2-oxoethyl]-4, 5, 6, 7-tetrahydrothieno [3, 2-c] pyridine-2-yl acetate OR 2[2-(acetyloxy)-6, 7-dihydrothieno [3, 2-c] pyridin-5(4H)-yl-]-1-cyclopropyl-2-(2flurophenyl) ethenone hydrochloride salt, having molecular formula C₂₀H₂₃FNO₃SCl and molecular weight 411.95.

Prasugrel HCl is a member of the thienopyridine class of ADP receptor inhibitors, like ticlopidine and clopidogrel (trade name Plavix). These agents are believed to reduce the aggregation (‘clumping’) of platelets by irreversibly binding to P2Y₁₂ receptors.

Prasugrel is first disclosed in U.S. Pat. No. 5,288,726 which also discusses its process for preparation which comprises i) condensing 2-oxo-2, 4, 5, 6, 7, 7a-hexahydrothieno [3, 2-c] pyridine or its salt with 2-chloro-a-cyclopropylcarbonyl benzyl bromide to obtain 5-(a-cyclopropylcarbonyl-2-fluorobenzyl)-2-oxo-2, 4, 5, 6, 7, 7a-hexahydrothieno [3, 2-c] pyridine and ii) reacting 5-(a-cy-clopropylcarbonyl-2-fluorobenzyl)-2-oxo-2, 4, 5, 6, 7, 7a-hexahydrothieno [3, 2-c] pyridine with acetic anhydride. This patent discloses preparation of hydrochloride salt of compounds analogues to Prasugrel by purging HCl gas in a solution of compound dissolved in Ether. A major drawback of this process is use of ether solvent as ether solvents are highly flammable, difficult to recover and hazardous to human health if inhale.

U.S. Pat. No. 6,693,115 disclose preparation of hydrochloride salt of Prasugrel by addition of conc. hydrochloric acid to solution of Prasugrel base in inert solvent. This process is silent about desacetyl prasugrel impurity which remains adhered with prasugrel HCl.

Impurities introduced during commercial manufacturing processes must be limited to very small amounts and are preferably substantially absent. For example, the ICH Q7A guidance for API manufacturers requires that process impurities be maintained below set limits by specifying the quality of raw materials, controlling process parameters, such as temperature, pressure, time, and stoichiometric ratios, and including purification steps, such as crystallization, distillation, and liquid-liquid extraction, in the manufacturing process.

It is therefore a need to develop a process for the preparation of Prasugrel HCl which overcomes the disadvantages associated with prior art processes as well as applicable at an industrial scale.

Surprisingly, present inventor found a process for the preparation of Prasugrel HCl which involves selective solvent system materially affects the quality and helps for overcoming disadvantages associated with prior art processes as well as applicable at an industrial scale.

### OBJECT OF THE INVENTION

It is therefore an object of the present invention is to provide process for the preparation Prasugrel HCl.

Another object of the present invention is to provide process for the preparation of Prasugrel HCl which is operationally simple, easy to handle and applicable at an industrial scale.

Yet another object of the present invention is to provide process for the preparation of Prasugrel HCl comprising:

i) preparing solution of HCl in alcohol

ii) preparing solution of Prasugrel base in ketone

iii) adding solution obtained in step (i) to a solution of step (ii)

Further object of the present invention is to provide process for the preparation of Prasugrel HCl comprising:

i) preparing solution of HCl in Isopropanol

ii) preparing solution of Prasugrel base in acetone

iii) adding solution obtained in step (i) to a solution of step (ii)

Further object of the present invention is to provide a process for the preparation of Prasugrel HCl comprising:

i) preparing solution of HCl in Isopropanol

ii) preparing solution of Prasugrel base in MEK

iii) adding solution obtained in step (i) to a solution of step (ii)

### SUMMARY OF THE INVENTION

In one aspect of the present invention, it provides a process for the preparation of Prasugrel HCl comprising:

i) preparing solution of HCl in alcohol

ii) preparing solution of Prasugrel base in ketone

iii) adding solution obtained in step (i) to a solution of step (ii)

### BRIEF DESCRIPTION OF DRAWINGS

FIG. 1. depicted characteristic powder XRD pattern of Prasugrel HCl obtained according to process of present invention.

FIG. 2. depicted characteristic IR spectrum of Prasugrel HCl obtained according to process of present invention.

FIG. 3 depicts characteristic differential scanning calorimetric (DSC) thermogram of Prasugrel HCl obtained according to process of present invention.

### DETAIL DESCRIPTION OF THE INVENTION

Accordingly, present invention describes process for the preparation of Prasugrel HCl comprising:

i) preparing solution of HCl in alcohol

ii) preparing solution of Prasugrel base in ketone

iii) adding solution obtained in step (i) to a solution of step (ii)

Deacetyl prasugrel impurity having formula II as mentioned hereinabove always formed in large amount during the preparation of Prasugrel HCl form prasugrel base.

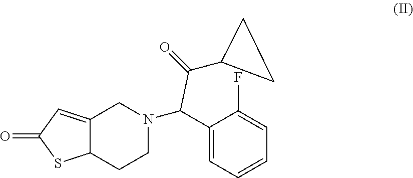 [SMILES: O=C1C=C2CN(C(C(=O)C3CC3)c3ccccc3F)CCC2S1]

Therefore, the large amount of contamination of this impurity is always adhered with the final product i.e. prasugrel HCl. This impurity was controlled by utilizing process of present invention which employs particular selective solvent system.

For the purpose of present invention, the meaning of the term ‘Ketone’ includes but not limited to symmetric and asymmetric ketonic solvents such as acetone, methyl ethyl ketone, methyl isobutyl ketone, isobutyl ketone and the like or mixture thereof. The preferred one is acetone and methyl ethyl ketone.

For the purpose of present invention, the meaning of the term ‘alcohol’ includes but not limited to alcoholic solvents such as methanol, ethanol, isopropanol, n-butanol, isobutanol and the like or mixture thereof. The preferred one is isopropanol.

Solution of HCl in alcohol preferably Isopropanol (IPA: HCl) used in process of present invention was prepared by purging HCl gas in alcohol preferably Isopropanol.

The solution of prasugrel base in ketone is prepared by dissolving prasugrel base in ketone. If required heat the mixture for the dissolution and obtain a solution. Optionally filtered this solution through hyflow.

The solution of HCl in alcohol was added in solution of Prasugrel base in ketone at temperature 30-50° C. The mixture was stirred at same temperature to obtain solid which was filtered and dried in tray dryer at 50-55° C. under vacuum to obtain prasugrel HCl.

Prasugrel base utilized in the process of present invention was prepared according to methods known in the prior art. These methods are also exemplified herein.

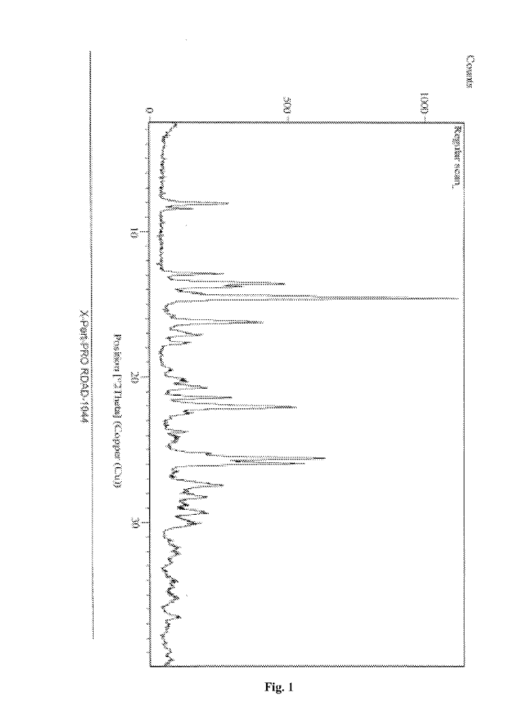

Prasugrel HCl obtained according to process of present invention shows XRD pattern as depicted in FIG. 1 which has been characterized by an X-ray powder diffraction spectrum having peaks at about 8.16, 8.51, 12.97, 13.64, 13.83, 14.25, 14.66, 16.35, 17.12, 17.68, 19.58, 20.35, 20.80, 21.51, 22.15, 22.59, 23.83, 24.15, 24.61, 25.66, 26.08, 27.49, 28.35, 29.41, 30.22, 36.59±0.2 degree two-theta. The XRD characteristic is obtained according to process of present invention is identical with prasugrel HCl form B2 as disclosed in known art.

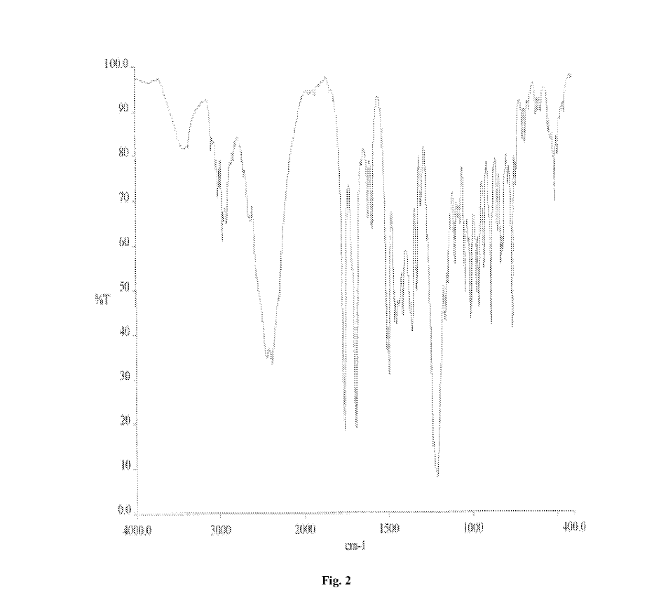

Prasugrel HCl obtained according to process of present invention shows IR spectrum pattern as depicted in FIG. 2 which has been characterized by an IR spectrum having peaks at about 3835.9, 3409.8, 3084.9, 3066.8, 3004.1, 2985.8, 2955.8, 2939.3, 2908.6, 2818.7, 2789.9, 2698.3, 2615.8, 2435.0, 2376.1, 1925.8, 1757.4, 1688.8, 1613.9, 1586.2, 1503.7, 1492.7, 1455.9, 1444.8, 1433.4, 1406.3, 1364.9, 1353.8, 1325.1, 1298.3, 1263.8, 1232.7, 1211.7, 1155.6, 1140.9, 1097.6, 1078.7, 1064.4, 1034.4, 1016.9, 1002.9, 969.2, 954.6, 923.8, 882.1, 843.1, 824.1, 811.3, 780.9, 756.9, 654.0, 613.2, 540.2, 529.0, 501.5, 485.8, 445.2 cm⁻¹. The IR characteristic is obtained according to process of present invention is identical with prasugrel HCl form B2 as disclosed in known art.

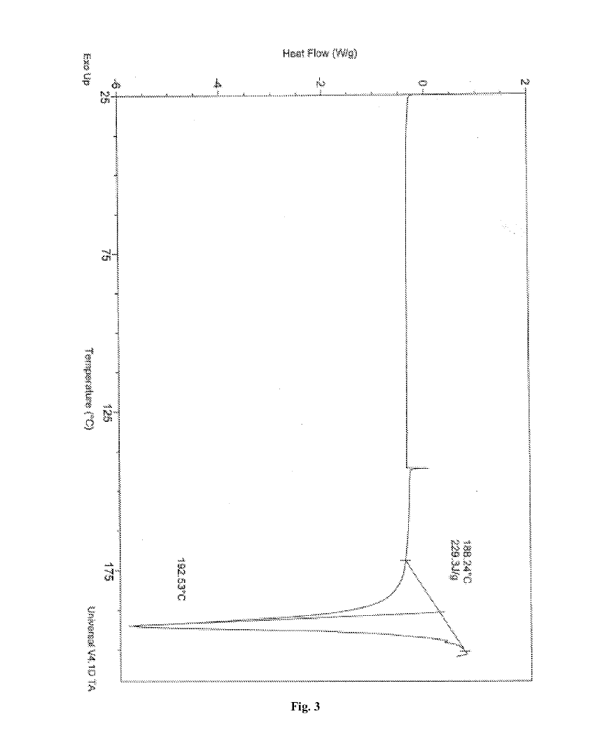

Prasugrel HCl obtained according to process of present invention shows DSC pattern as depicted in FIG. 3 characterized by differential scanning calorimetry (DSC) thermogram endotherm peak occurs in the temperature range from about 191° C. to about 194° C. and normally occurs at 192° C.

The surface area per unit weight, which is known as specific surface, is increased by size reduction. This increased specific surface affects the therapeutic efficacy of pharmaceutical compounds that possess a low solubility in body fluids by increasing the area of contact between the solid and the dissolving fluid. Thus, a given weight of finely powdered pharmaceutical compounds dissolves in a shorter time than does same weight of a coarser powder.

Materials used in pharmaceuticals must be existing in the optimum size and most materials must be communicated at some stage during the production of dosage form. Milling is the mechanical process reducing the particle size of pharmaceutical solids. Various terms like crushing, disintegration, dispersion, grinding and pulverization used synonymously. These processes have been used for reduction of particle size of solids in pharmaceutical industry routinely.

Particle size is believed to be an important parameter affecting clinical effectiveness of Prasugrel HCl. Therefore, composition of Prasugrel HCl obtained according to process of present invention may be milled or grind. Prasugrel HCl obtained according to process of present invention may have particle sizes distribution such that,

i) D₉₀ of the particles is less 200 μm, D₅₀ of the particles is less 100 μm and D₁₀ of the particles is less 50 μm or,

ii) D₉₀ of the particles is less 100 μm, D₅₀ of the particles is less 50 μm and D₁₀ of the particles is less 10 μm or,

iii) D₉₀ of the particles is less 200 μm or,

iv) D₅₀ of the particles is less 100 μm or,

v) D₁₀ of the particles is less 50 μm

Prasugrel HCl obtained according to process of present invention having particle size distribution such that D₉₀ of the particles is less 200 μm.

XRD was recorded by X-pert-PRO RDAD-1044. DSC was recorded by Universal V4.1D TA Instruments. IR was recorded by Perkin Elmer Instruments.

Following are the advantages of process of present invention:

i) Providing consistently high purity prasugrel HCl with HPLC purity above 99%

ii) Controlling desacetyl prasugrel impurity

The following examples illustrate the invention further. It should be understood, however, that the invention is not confined to the specific limitations set forth in the individual examples but rather to the scope of the appended claims.

### Example-1

Preparation of 2-Oxo-2, 4, 5, 6, 7, 7a-hexahydro thieno [3, 2-c] pyridine hydrochloride

In to a 2.0 lit RBF, 5-triphenylmethyl- 2- oxo-2, 4, 5, 6, 7, 7a-hexahydro thieno [3, 2-c] pyridine (100 gm) was charged in acetone (1500 ml) at the room temperature to obtain reaction mixture. Then conc. hydrochloric acid (24.87 ml) was added to reaction mixture & heated at 55-60° C. for 3 hrs. After completing reaction mixture, reaction mixture was cooled at room temperature to crystallize out a compound. The compound was filtered and washed with acetone and dried under vacuum at 60-65° C. to obtain title compound (35-45 gm).

### Example-2

Preparation of 2-Acetoxy-5-(α-cyclopropylcarbonyl-2-fluorobenzyl) -4, 5, 6, 7-tetrahydrothieno [3, 2-c] pyridine (crude Prasugrel)

100 gm of 2-Oxo-2, 4, 5, 6, 7, 7a-hexahydro thieno [3, 2-c] pyridine hydrochloride was charged in 800 ml of dimethylformamide to obtain reaction mixture. To this sodium bicarbonate (175 gm) and a -Cyclopropyl carbonyl-2-fluorobenzylbromide (175 gm) was added at room temperature and allowed to heat at 50-55° C. for 2 hrs. The reaction mixture was cooled up to 10-15° C. Acetic anhydride (118.67 gm) was added drop by drop at same temperature. The reaction mixture was heated to 40-45° C. for 3 hrs & cooled the reaction mixture to room temperature. Ethyl acetate and water was added to reaction mixture. The organic layer was separated and aqueous layer was discarded. The organic ethyl acetate layer was washed by aq. Na₂CO₃ followed by aq. NaCl solution. The ethyl layer was distilled of completely to obtain product mass. Methanol was charged in the obtained mass and stirred it at room temperature & cooled up to 0-5° C. to obtain solid which was filtered and washed with methanol and dried at 50-55° C. under vacuum to obtain crude prasugrel (85-95 gm).

### Example-3

Purification of 2-Acetoxy-5-(α-cyclopropylcarbonyl-2-fluorobenzyl)-4, 5, 6, 7-tetrahydrothieno [3, 2-c] pyridine (Pure Prasugrel)

100 gm of Prasugrel was charged in 500 ml of acetone in a clean & dry RBF at room temperature. Then maleic acid (32 gm) was added to it. The reaction mixture was heated up to 30-35° C. for 2-4 hrs. After completion of reaction, the reaction mixture was cooled to 25-30° C. and maintains the reaction mixture for 30 min to obtain solid which was filtered and washed by acetone to obtain wet maleate salt. In another flask, sodium bicarbonate (49.34 gm) & water (1250 ml) was charged to obtain mixture. To this mixture, ethyl acetate (1400 ml) and wet cake of maleate salt was added room temperature and stirred it for 1 hr at 25-30° C. Reaction mix was allowed to settle for 15-30 min. The layer was separated at room temperature to obtain the product in organic ethyl acetate layer which was washed by aq. Na₂CO₃ followed by aq. NaCl solution. The organic ethyl layer was distilled of completely to obtain product mass. Isopropanol (100 ml) was added to product mass & distilled out isopropanol under vacuum at 35-40° C. to obtain solid. Isopropanol was added for dissolution of solid completely at 75-80° C. to obtain clear solution. This solution was cooled to 25-30° C. and stirred to obtain solid which was filtered washed with isopropanol dried at 50-55° C. under vacuum to obtain Prasugrel (70-90 gm).

### Example-4

Preparation of Prasugrel Hydrochloride

100 gm of Pure Prasugrel was charged in methyl ethyl ketone (800 ml) in a clean & dry RBF. This reaction mass was stirred at room temperature for 30 min. and filtered through hyflow followed by washing hyflow bed with methyl ethyl ketone (200 ml) to obtain filtrate. The filtrate was heated up to 35-40° C. to obtain reaction mixture. To this reaction mixture, IPA: HCl (33.5 ml) solution (solution prepared by purging HCl gas in isopropanol) was charged at 35-40° C. in 20 min. The reaction mixture was maintained at 35-40° C. for 60 min to obtain solid. The solid was filtered and washed with methyl ethyl ketone and dried in tray dryer at 50-55° C. under vacuum to get prasugrel HCl (70-90 gm) polymorphic form B2.

HPLC purity˜above 99%

Desacetyl prasugrel impurity˜less than 0.1%

1) A process for the preparation of Prasugrel HCl comprising:
i) preparing a solution of HCl in an alcohol;
ii) preparing a solution of Prasugrel base in a ketone; and
iii) adding the solution obtained in step (i) to the solution of step (ii).

2) The process according to claim 1, wherein the alcohol is selected from the group consisting of methanol, ethanol, isopropanol, n-butanol, isobutanol, and mixtures thereof.

3) The process according to claim 1, wherein the ketone is selected from the group consisting of acetone, methyl ethyl ketone, methyl isobutyl ketone, isobutyl ketone, and mixtures thereof.

4) A process for the preparation of Prasugrel HCl comprising:
i) preparing a solution of HCl in isopropanol;
ii) preparing a solution of Prasugrel base in methyl ethyl ketone; and
iii) adding the solution obtained in step (i) to the solution of step (ii).

5) The process according to claim 4, wherein the solution of HCl in isopropanol is prepared by purging HCl gas in isopropanol.

6) The process according to claim 4, wherein the solution obtained in step (i) is added to the solution of step (ii) at a temperature of 30-45° C.

7) Prasugrel HCl having a X-ray powder diffraction spectrum having peaks at about 8.16, 8.51, 12.97, 13.64, 13.83, 14.25, 14.66, 16.35, 17.12, 17.68, 19.58, 20.35, 20.80, 21.51, 22.15, 22.59, 23.83, 24.15, 24.61, 25.66, 26.08, 27.49, 28.35, 29.41, 30.22, and 36.59±0.2 degree two-theta, and having less than 0.1% of an impurity of the formula,

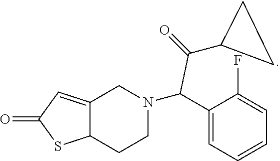 [SMILES: O=C1C=C2CN(C(C(=O)C3CC3)c3ccccc3F)CCC2S1]

8) Prasugrel HCl having an IR spectrum having peaks at about 3835.9, 3409.8, 3084.9, 3066.8, 3004.1, 2985.8, 2955.8, 2939.3, 2908.6, 2818.7, 2789.9, 2698.3, 2615.8, 2435.0, 2376.1, 1925.8, 1757.4, 1688.8, 1613.9, 1586.2, 1503.7, 1492.7, 1455.9, 1444.8, 1433.4, 1406.3, 1364.9, 1353.8, 1325.1, 1298.3, 1263.8, 1232.7, 1211.7, 1155.6, 1140.9, 1097.6, 1078.7, 1064.4, 1034.4, 1016.9, 1002.9, 969.2, 954.6, 923.8, 882.1, 843.1, 824.1, 811.3, 780.9, 756.9, 654.0, 613.2, 540.2, 529.0, 501.5, 485.8, and 445.2 cm⁻¹, and having less than 0.1% of an impurity of the formula,

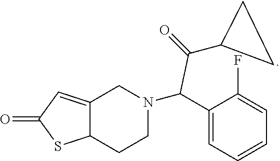 [SMILES: O=C1C=C2CN(C(C(=O)C3CC3)c3ccccc3F)CCC2S1]

9) Prasugrel HCl having a differential scanning calorimetry (DSC) having a thermogram endotherm peak in a temperature range from about 191° C. to about 194° C., and having less than 0.1% of an impurity of the formula,

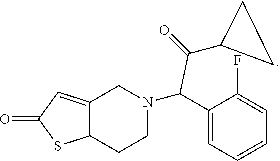 [SMILES: O=C1C=C2CN(C(C(=O)C3CC3)c3ccccc3F)CCC2S1]

10) Prasugrel HCl having a particle size distribution such that D₉₀ of the particles is less than 200 μm, and having less than 0.1% of an impurity of the formula,

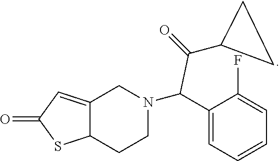 [SMILES: O=C1C=C2CN(C(C(=O)C3CC3)c3ccccc3F)CCC2S1]

11) The process according to claim 1, wherein the alcohol is isopropanol and the ketone is acetone or methyl ethyl ketone.

12) The process according to claim 1, wherein the solution of HCl in an alcohol is prepared by purging HCl gas in the alcohol.

13) The process according to claim 1, wherein the solution obtained in step (i) is added to the solution of step (ii) at a temperature of 30-50° C.

14) The process according to claim 1, further comprising filtering a solid product obtained from step (iii); drying the solid product; and milling or grinding the dried solid product, wherein the dried solid product is milled or ground to have a particle size distribution, wherein
(a) D₉₀ of the particles is less than 200 μm, D₅₀ of the particles is less than 100 μm and D₁₀ of the particles is less than 50 μm;
(b) D₉₀ of the particles is less than 100 μm, D₅₀ of the particles is less than 50 μm and D₁₀ of the particles is less than 10 μm;
(c) D₉₀ of the particles is less than 200 μm;
(d) D₅₀ of the particles is less than 100 μm; or
(e) D₁₀ of the particles is less than 50 μm.

15) The process according to claim 4, further comprising filtering a solid product obtained from step (iii); drying the solid product; and milling or grinding the dried solid product, wherein the dried solid product is milled or ground to have a particle size distribution, wherein
(a) D₉₀ of the particles is less than 200 μm, D₅₀ of the particles is less than 100 μm and D₁₀ of the particles is less than 50 μm;
(b) D₉₀ of the particles is less than 100 μm, D₅₀ of the particles is less than 50 μm and D₁₀ of the particles is less than 10 μm;
(c) D₉₀ of the particles is less than 200 μm;
(d) D₅₀ of the particles is less than 100 μm; or
(e) D₁₀ of the particles is less than 50 μm.

16) A process for the preparation of Prasugrel HCl comprising adding a solution of HCl in an alcohol to a solution of Prasugrel base in a ketone, wherein the HCl solution is added at a temperature of 30-50° C.

17) The process according to claim 16, wherein the alcohol is selected from the group consisting of methanol, ethanol, isopropanol, n-butanol, isobutanol, and mixtures thereof.

18) The process according to claim 16, wherein the ketone is selected from the group consisting of acetone, methyl ethyl ketone, methyl isobutyl ketone, isobutyl ketone, and mixtures thereof.

19) The process of claim 16, wherein the alcohol is isopropanol and the ketone is methyl ethyl ketone.

20) Prasugrel HCl having less than 0.1% of an impurity of the formula, 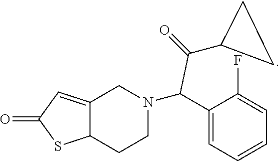 [SMILES: O=C1C=C2CN(C(C(=O)C3CC3)c3ccccc3F)CCC2S1]
